(19) United States
(12) Patent Application Publication
(10) Pub. No.: US 2012/0209084 A1
(43) Pub. Date: Aug. 16, 2012

(54) RESPIRATORY EVENT ALERT SYSTEM

(75) Inventors: Greg A. Olsen, Trabuco Canyon, CA (US); Phil Weber, Solano Beach, CA (US)

(73) Assignee: Masimo Corporation, Irvine, CA (US)

(21) Appl. No.: 13/355,404

(22) Filed: Jan. 20, 2012

Related U.S. Application Data
(60) Provisional application No. 61/435,130, filed on Jan. 21, 2011.

(51) Int. Cl. A61B 5/0205; A61B 5/00; A61B 7/00; A61B 5/08

(52) U.S. Cl. 600/301; 600/529

(57) ABSTRACT

A respiratory monitoring system can include an acoustic respiratory sensor that obtains acoustic information from a patient and one or more processors in communication with the acoustic respiratory sensor. Such processors can receive a respiratory rate value derived from the acoustic information. This respiratory rate value can reflect an averaged set of respiratory rate values over a period of time. The one or more processors can also receive an indication of a respiratory abnormality that occurred during the same time period. Further, the one or more processors can output the averaged respiratory rate value together with a respiratory event indicator reflecting the respiratory abnormality.

### CROSS REFERENCE TO RELATED APPLICATION

This application is a non-provisional of U.S. Provisional Patent Application No. 61/435,130, filed Jan. 21, 2011, which is hereby incorporated by reference herein in its entirety.

### BACKGROUND

Hospitals, nursing homes, and other patient care facilities typically include patient monitoring devices at one or more bedsides in the facility. Patient monitoring devices generally include sensors, processing equipment, and displays for obtaining and analyzing a patient's physiological parameters. Physiological parameters include, for example, blood pressure, respiratory rate, oxygen saturation (SpO₂) level, other blood constitutions and combinations of constitutions, and pulse, among others. Clinicians, including doctors, nurses, and certain other caregiver personnel use the physiological parameters obtained from the patient to diagnose illnesses and to prescribe treatments. Clinicians can also use the physiological parameters to monitor a patient during various clinical situations to determine whether to increase the level of care given to the patient. Various patient monitoring devices are commercially available from Masimo Corporation (“Masimo”) of Irvine, Calif.

During and after surgery and in other care situations, respiratory rate is a frequently monitored physiological parameter of a patient. Respiratory rate can be indicated as the number of breaths a person takes within a certain amount of time, such as breaths per minute. For example, a clinician (such as a nurse, doctor, or the like) can use respiratory rate measurements to determine whether a patient is experiencing respiratory distress and/or dysfunction.

### SUMMARY OF DISCLOSURE

In certain embodiments, a respiratory monitoring system for indicating respiratory abnormalities on a physiological monitor display includes an acoustic respiratory sensor configured to obtain acoustic information from a patient. The acoustic information can reflect one or more physiological parameters of the patient. The system can further include one or more processors in communication with the acoustic respiratory sensor. The one or more processors can be configured to: receive a respiratory rate value, the respiratory rate value reflecting a respiratory measurement obtained from the acoustic information averaged over a time period; receive an indication of a respiratory abnormality occurring during the time period; and output the respiratory rate value together with a respiratory event indicator reflecting the respiratory abnormality.

In various embodiments, a method for indicating respiratory abnormalities on a physiological monitor display can be performed by one or more processors. The one or more processors can receive a respiratory rate value from a respiratory rate calculator, the respiratory rate value corresponding to respiration of a patient averaged over a time period, such that the respiratory rate value does not directly indicate abnormal respiration in the patient. The one or more processors can further output the respiratory value on a physiological monitor display for presentation to a clinician. Moreover, the one or more processors can receive an indication of a respiratory abnormality occurring during the time period. Additionally, the one or more processors can output an indicator reflecting the respiratory abnormality on the physiological monitor display.

For purposes of summarizing the disclosure, certain aspects, advantages and novel features of the inventions have been described herein. It is to be understood that not necessarily all such advantages can be achieved in accordance with any particular embodiment of the inventions disclosed herein. Thus, the inventions disclosed herein can be embodied or carried out in a manner that achieves or optimizes one advantage or group of advantages as taught herein without necessarily achieving other advantages as can be taught or suggested herein.

### BRIEF DESCRIPTION OF THE DRAWINGS

Throughout the drawings, reference numbers can be re-used to indicate correspondence between referenced elements. The drawings are provided to illustrate embodiments of the inventions described herein and not to limit the scope thereof.

FIG. 1 is a block diagram illustrating an embodiment of a patient monitoring system;

FIG. 2 is a top perspective view illustrating portions of an example sensor assembly that can be used with the patient monitoring system of FIG. 1;

FIG. 3 is a block diagram illustrating an embodiment of a respiratory monitoring system;

FIG. 4 illustrates an example display of respiratory trend data and respiratory event indicators provided by the respiratory monitoring system of FIG. 3;

FIG. 5 illustrates an embodiment of a process for displaying respiratory data;

FIG. 6A illustrates another embodiment of a respiratory monitoring system;

FIG. 6B illustrates an embodiment of a process for displaying respiratory data provided by the respiratory monitoring system of FIG. 6A;

FIGS. 7 and 8 illustrate example displays of a respiratory monitoring system of FIG. 6A;

FIG. 9 illustrates a rating system that indicates both a calculated confidence level in a detection of a respiratory event and an estimated severity of the respiratory event.

### DETAILED DESCRIPTION

Acoustic sensors, including piezoelectric acoustic sensors, can be used to measure breath sounds and other biological sounds of a patient. Breath sounds obtained from an acoustic sensor can be processed by a patient monitor to derive one or more physiological parameters of a patient, including respiratory rate. Respiratory rate can also be measured using other instruments, including a capnograph, which can measure respiratory rate from an end-tidal carbon dioxide (EtCO₂) waveform, and a pulse oximeter, which can derive respiratory rate from a photoplethysmograph. However, respiratory rate measurements can be an incomplete representation of a patient's breathing events.

Currently-available respiratory monitors provide respiratory indicators, such as a respiratory rate. Because data used to compute respiratory rate can be noisy and/or vary over time, respiratory rate is typically computed as an average of breaths taken over a certain time frame. However, providing averaged respiratory rate data alone may not capture respiratory events of clinical interest. For example, a patient with a respiratory rate (RR) of 20 breaths per minute averaged over 30 seconds can subsequently have a 30 second apnea. In such a case, the monitor would show a RR of 10 breaths per minute. In this example, the monitor would not directly output an indication regarding the 30 second lack of breathing.

Advantageously, the respiratory monitoring systems described herein can detect respiratory events and provide indicators of the respiratory events along with respiratory rate data. Such indicators of respiratory events can also convey specific information about the respiratory event. For example, an indicator of a respiratory event can convey an estimated severity of a respiratory event, length of occurrence of such an event, and/or a calculated confidence level in a detection of the respiratory event. Respiratory event indicators can provide a clinician with an indication that the respiratory event has been tracked and should be observed.

This disclosure describes, among other features, systems and methods for using one or more physiological parameter inputs to calculate respiratory rate and detect respiratory events. Respiratory events can include such respiratory abnormalities such as increased respiration events, reduced respiration events, and respiratory pause events (e.g., non-respiration events). Some example respiratory events include, but are not limited to, apnea, obstruction, wheezes, striders, ronchi, rales, hypopnea, dyspnea, bradypnea, and decreased volume or change in airflow. In certain embodiments, a patient monitoring system can output an indicator of a respiratory event that can convey one or more characteristics of the respiratory event, along with respiratory rate data, for presentation to a clinician.

For purposes of illustration, this disclosure is described primarily in the context of respiratory rate. However, the features described herein can be applied in the context of other respiratory parameters, including, for example, inspiratory time, expiratory time, inspiratory to expiratory ratio, inspiratory flow, expiratory flow, tidal volume, minute volume, changes in breath sounds, and the like.

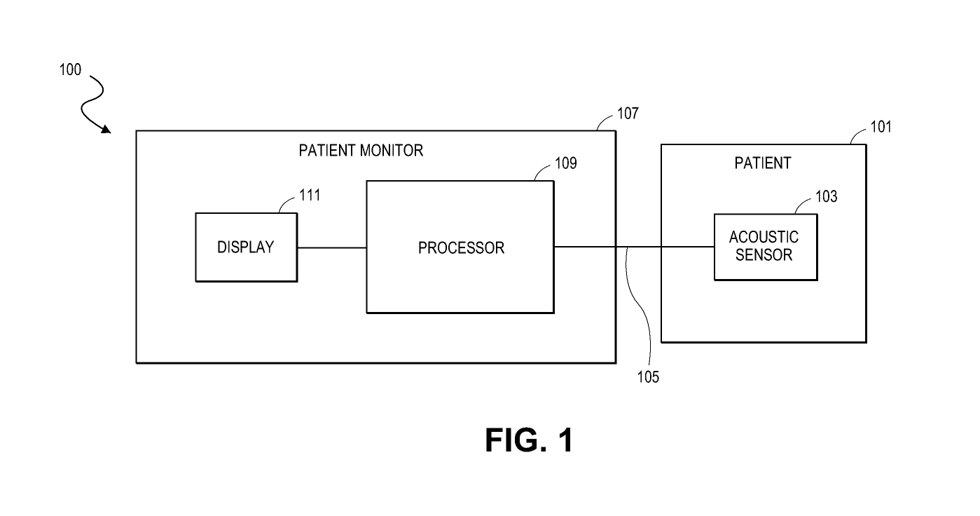

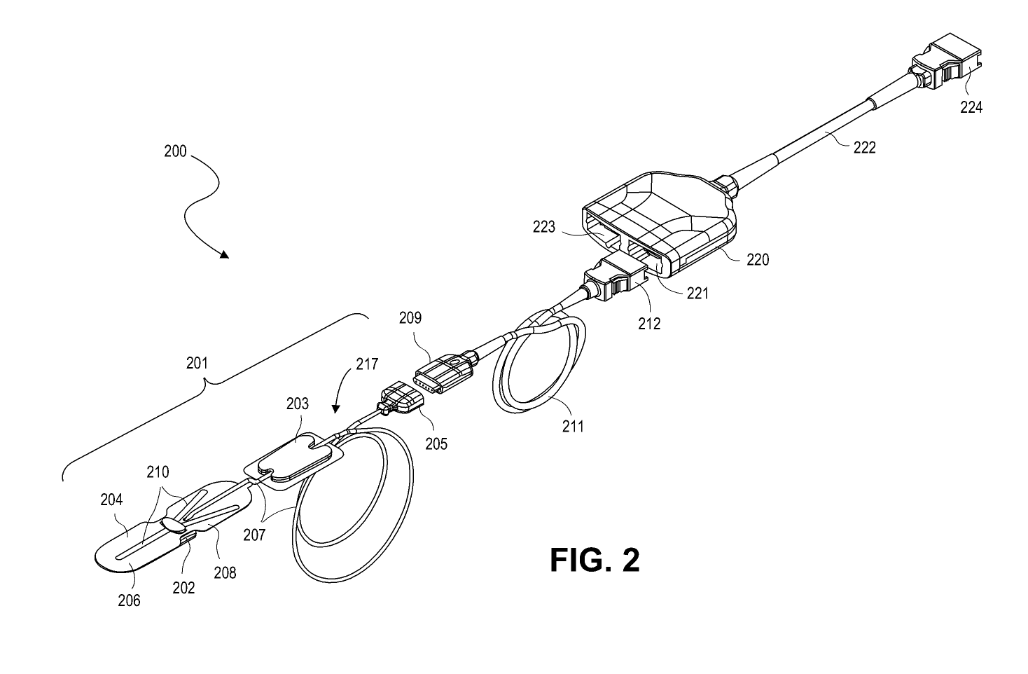

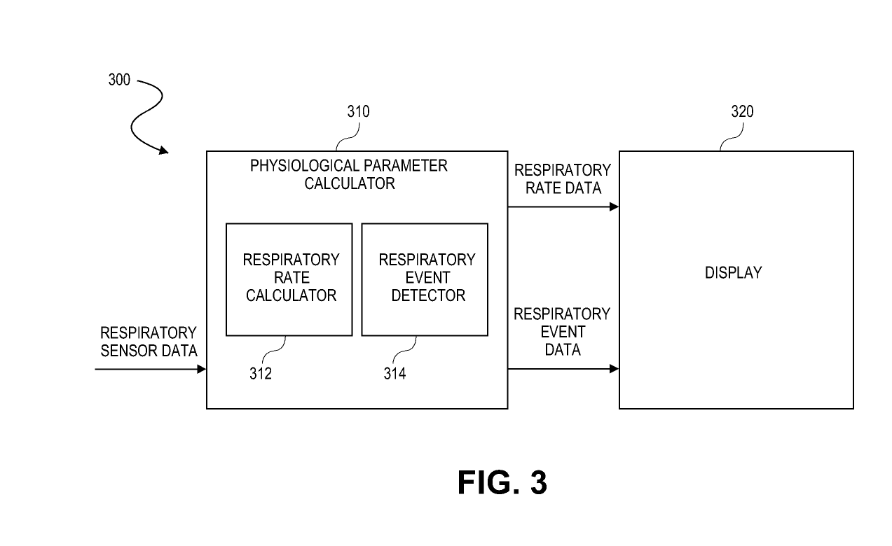

Referring to the drawings, FIGS. 1 and 2 illustrate example patient monitoring systems, sensors, and cables that can be used to derive a respiratory rate measurement from a patient. FIGS. 3 through 9 illustrate respiratory monitoring systems, along with associated displays and processes. The features of FIGS. 3 through 9 can be implemented at least in part using the systems and sensors described in FIGS. 1 and 2.

With reference to FIG. 1, an embodiment of a physiological monitoring system 100 is shown. In the physiological monitoring system 100, a patient 101 can be monitored using one or more acoustic sensor assemblies 103, each of which can transmit one or more signals over a cable 105 or other communication link or medium to a physiological monitor 107. The physiological monitor 107 can also be referred to as a patient monitor. The physiological monitor 107 can include a processor 109 and, optionally, a display 111. The one or more acoustic sensors 103 include sensing elements such as, for example, acoustic piezoelectric devices or the like. The acoustic sensors 103 can generate respective signals by measuring a physiological parameter of the patient 101. The signals can then be processed by one or more processors 109. The one or more processors 109 can then communicate the processed signal to the display 111. In an embodiment, the display 111 is incorporated in the physiological monitor 107. In another embodiment, the display 111 is separate from the physiological monitor 107. In one embodiment, the monitoring system 100 is a portable monitoring system. In another embodiment, the monitoring system 100 is a pod, without a display, that is adapted to provide physiological parameter data to a display.

For clarity, a single block is used to illustrate the one or more acoustic sensors 103 shown in FIG. 1. It should be understood that the acoustic sensor 103 shown is intended to represent one or more sensors. In an embodiment, the one or more acoustic sensors 103 include a single sensor of one of the types described below. In another embodiment, the one or more acoustic sensors 103 include at least two acoustic sensors. Other combinations of numbers and types of sensors are also suitable for use with the physiological monitoring system 100.

In some embodiments of the system shown in FIG. 1, all of the hardware used to receive and process signals from the sensors are housed within the same housing. In other embodiments, some of the hardware used to receive and process signals is housed within a separate housing. In addition, the physiological monitor 107 of certain embodiments includes hardware, software, or both hardware and software, whether in one housing or multiple housings, used to receive and process the signals transmitted by the sensors 103.

FIG. 2 illustrates an embodiment of a sensor system 200 including a sensor assembly 201 and a monitor cable 211 suitable for use with the physiological monitor shown in FIG. 1. For example, the sensor system 200 can implement the acoustic sensor 103 (FIG. 1) that can be used to acquire respiratory data. The sensor assembly 201 can include a sensor 215, a cable assembly 217, and a connector 205. The sensor 215, in one embodiment, includes a sensor subassembly 202 and an attachment subassembly 204. The cable assembly 217 of one embodiment includes a sensor cable 207 and a patient anchor 203. A sensor connector subassembly 205 can be connected to the sensor cable 207.

The sensor connector subassembly 205 can be removably attached to an instrument cable 211 via an instrument cable connector 209. The instrument cable 211 can be attached to a cable hub 220, which can include a port 221 for receiving a connector 212 of the instrument cable 211 and a second port 223 for receiving another cable. In certain embodiments, the second port 223 can receive a cable connected to a pulse oximetry or other sensor. In addition, the cable hub 220 can include additional ports in other embodiments for receiving additional cables. The hub can include a cable 222 which terminates in a connector 224 adapted to connect to a physiological monitor (not shown).

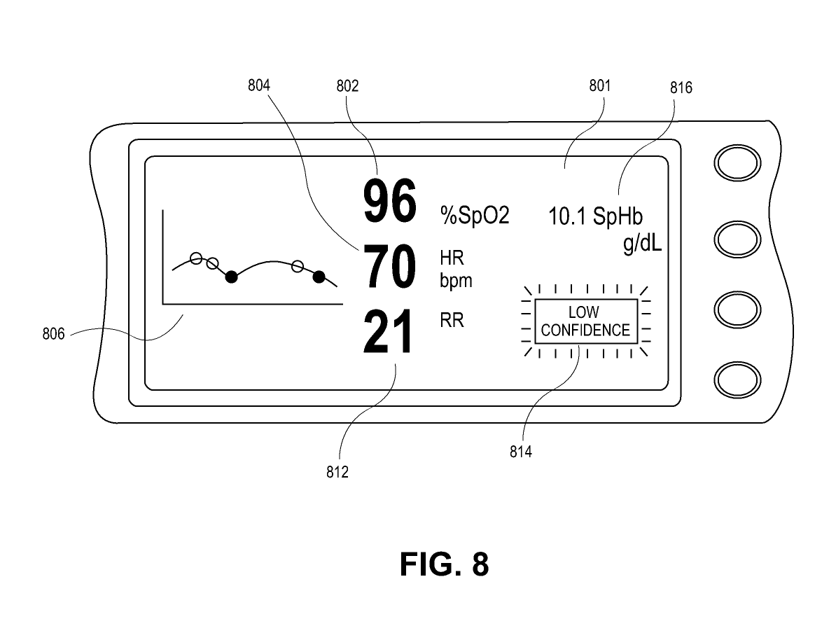

The sensor connector subassembly 205 and connector 209 can allow the sensor connector 205 to be straightforwardly and efficiently joined with and detached from the connector 209. Embodiments of connectors having connection mechanisms that can be used for the connectors 205, 209 are described in U.S. patent application Ser. No. 12/248,856 (hereinafter referred to as “the '856 application”), filed on Oct. 9, 2008, which is incorporated in its entirety by reference herein. For example, the sensor connector 205 could include a mating feature (not shown) which mates with a corresponding feature (not shown) on the connector 209. The mating feature can include a protrusion which engages in a snap fit with a recess on the connector 209. In certain embodiments, the sensor connector 205 can be detached via one hand operation, for example. Examples of connection mechanisms can be found specifically in paragraphs [0042], [0050], [0051], [0061]-[0068] and [0079], and with respect to FIGS. 8A-F, 13A-E, 19A-F, 23A-D and 24A-C of the '856 application, for example.

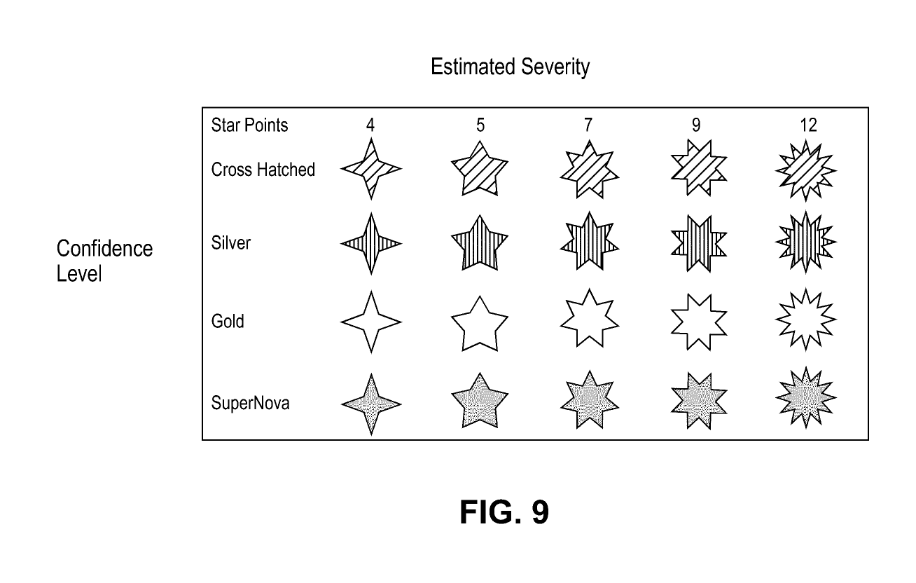

The sensor connector subassembly 205 and connector 209 can reduce the amount of unshielded area in and generally provide enhanced shielding of the electrical connection between the sensor and monitor in certain embodiments. Examples of such shielding mechanisms are disclosed in the '856 application in paragraphs [0043]-[0053], [0060] and with respect to FIGS. 9A-C, 11A-E, 13A-E, 14A-B, 15A-C, and 16A-E, for example.

In an embodiment, the acoustic sensor assembly 201 includes a sensing element, such as, for example, a piezoelectric device or other acoustic sensing device. The sensing element can generate a voltage that is responsive to vibrations generated by the patient, and the sensor can include circuitry to transmit the voltage generated by the sensing element to a processor for processing. In an embodiment, the acoustic sensor assembly 201 can include circuitry for detecting and transmitting information related to biological sounds to a physiological monitor. These biological sounds can include heart, breathing, and/or digestive system sounds, in addition to many other physiological phenomena. The acoustic sensor 215 in certain embodiments is a biological sound sensor, such as the sensors described herein. In some embodiments, the biological sound sensor is one of the sensors such as those described in U.S. patent application Ser. No. 12/044,883, filed Mar. 7, 2008, entitled “Systems and Methods for Determining a Physiological Condition Using an Acoustic Monitor,” (hereinafter referred to as “the '883 application”), the disclosure of which is hereby incorporated by reference in its entirety. In other embodiments, the acoustic sensor 215 is a biological sound sensor such as those described in U.S. Pat. No. 6,661,161, which is incorporated by reference herein in its entirety. Other embodiments include other suitable acoustic sensors.

The attachment sub-assembly 204 can include first and second elongate portions 206, 208. The first and second elongate portions 206, 208 can include patient adhesive (e.g., in some embodiments, tape, glue, a suction device, etc.). The adhesive on the elongate portions 206, 208 can be used to secure the sensor subassembly 202 to a patient's skin. One or more elongate members 210 included in the first and/or second elongate portions 206, 208 can beneficially bias the sensor subassembly 202 in tension against the patient's skin and reduce stress on the connection between the patient adhesive and the skin. A removable backing can be provided with the patient adhesive to protect the adhesive surface prior to affixing to a patient's skin.

The sensor cable 207 can be electrically coupled to the sensor subassembly 202 via a printed circuit board (“PCB”) (not shown) in the sensor subassembly 202. Through this contact, electrical signals can be communicated from the multi-parameter sensor subassembly to the physiological monitor through the sensor cable 207 and the cable 211.

In various embodiments, not all of the components illustrated in FIG. 2 are included in the sensor system 200. For example, in various embodiments, one or more of the patient anchor 203 and the attachment subassembly 204 are not included. In one embodiment, for example, a bandage or tape is used instead of the attachment subassembly 204 to attach the sensor subassembly 202 to the measurement site. Moreover, such bandages or tapes can be a variety of different shapes including generally elongate, circular and oval, for example. In addition, the cable hub 220 need not be included in certain embodiments. For example, multiple cables from different sensors could connect to a monitor directly without using the cable hub 220.

Additional information relating to acoustic sensors compatible with embodiments described herein, including other embodiments of interfaces with the physiological monitor, are included in the '883 application. An example of an acoustic sensor that can be used with the embodiments described herein is disclosed in U.S. Patent Application No. 61/252,076, filed Oct. 15, 2009, titled “Acoustic Sensor Assembly,” the disclosure of which is hereby incorporated by reference in its entirety.

FIG. 3 is a block diagram illustrating an embodiment of a respiratory monitoring system 300. The respiratory monitoring system 300 can be used to process respiratory data obtained from one or more sensors, which can include, for example, the acoustic sensor 103 (FIG. 1), the sensor assembly 201 (FIG. 2), or any of the sensors described herein. The respiratory monitoring system 300 can be included as part of the physiological monitor 107 (FIG. 1). The illustrated respiratory monitoring system 300 includes a physiological parameter calculator 310 and a display 320.

The physiological parameter calculator 310 can be included, for example, as part of the processor 109 (FIG. 1). Respiratory data can be provided to the physiological parameter calculator 310. The physiological parameter calculator 310 can include a respiratory rate calculator 312 and a respiratory event detector 314. The respiratory rate calculator 312 can determine a respiratory rate based at least partly on the respiratory data. The respiratory event detector 314 can detect respiratory events and provide associated data. The physiological parameter calculator 310 can provide both respiratory rate data and respiratory event data to the display 320. Alternatively or additionally, the respiratory rate data and the respiratory event data can be provided to other user devices. The respiratory rate data and the respiratory event data can be provided though wires or over a network.

The respiratory rate calculator 312 can derive a respiratory rate from respiratory sensor data, for example, data provided by the sensor of FIG. 2. A breath can be detected by identifying certain features, such as a peak or trough, on a waveform of the respiratory data. Then a respiratory rate can be computed as the number of breaths detected in a certain time period. For example, a breath can be identified as a peak or trough that satisfies a predetermined threshold value. Respiratory rate can be expressed in breaths per minute. One example system for calculating respiratory rate, apnea, and other respiratory characteristics, which can be used with any of the embodiments described herein, is described in U.S. Publication No. 2007/0282212, filed Jun. 19, 2007, titled “Non-Invasive Monitoring of Respiratory Rate, Heart Rate, and Apnea,” attorney docket number MCAN.019NP, the disclosure of which is hereby incorporated by reference in its entirety.

Due to noisy respiratory data and/or variability in breathing, the respiratory rate can be averaged over time, such as 30 seconds, 60 seconds, or some other time period. Averaging can provide more robust and accurate respiratory data. At the same time, averaging respiratory rate over time can provide an incomplete representation of a patient's breathing events. For example, certain significant variations in respiratory rate within the averaging time may be lost. Such variations can reflect any of the respiratory abnormalities or events described herein.

The respiratory event detector 314 can detect one or more respiratory events from the respiratory sensor data. The one or more respiratory events can be respiratory abnormalities, increased respiration events, reduced respiration events, and/or non-respiration events. Example respiratory events can include, but are not limited to, apnea, obstruction, wheezes, striders, ronchi, rales, hypopnea, and breath sounds such as decreased volume or change in airflow. In some embodiments, the respiratory event detector 314 can provide data indicating a particular respiratory event.

Respiratory events can be detected a variety of ways. Detecting different events can include a variety of signal processing techniques. For example, reduced respiration events, such as apnea, can be identified by a cessation in breathing over a certain period of time. Other respiratory events can be detected by identifying patterns in breathing that are indicative of different respiratory problems. For example, apnea may be detected by a cessation of breathing for more than a predetermined period of time.

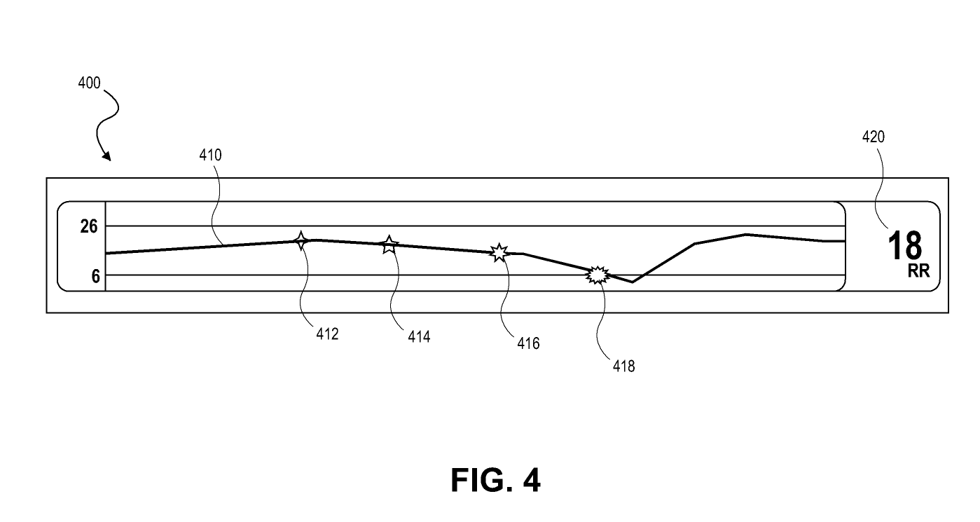

The respiratory event detector 314 can also determine characteristics of the detected respiratory event. Such characteristics can include an estimated severity of a respiratory event and/or a calculated confidence level in a detection of the respiratory event. The estimated severity of an event can be based on any indicator that a patient should require more or less medical attention. The estimated severity can be represented by, for example, a point indicator. A higher point indicator can be reflective of a higher severity and a lower point indicator can be reflective of a lower severity. More detail about how the estimated severity can be displayed will be provided below in connection with FIGS. 4 and 9.

The estimated severity of an event can be based on, for example, one or more of a duration of an event, a number of occurrences of the event within a predetermined time period, an intensity of an event as determined by features of the acoustic respiratory data, combinations of the same, or the like. Alternatively or additionally, the estimated severity of a respiratory event can be based at least partly on the type of respiratory event detected. For example, an apnea event can have a higher estimated severity than a hypopnea event. As another example, a rales event can have a lower estimated severity than an obstruction event.

The estimated severity of a respiratory event and/or confidence level in detecting the respiratory event can be presented as one or more respiratory event indicators to a clinician on a display 320. The display 320 can be part of and/or separate from the patient monitor, for example, the physiological monitor 107 (FIG. 1). The display can implement any of the features of the display 111 (FIG. 1).

The display 320 can present, among other things, a respiratory rate indicator, along with the one or more respiratory event indicators. The respiratory rate indicator can be represented by, for example, a trend graph, a set of discrete points, a number representing an average or moving average of a respiratory rate over a predetermined period of time, a series of numbers, or audio representations.

Each of the one or more respiratory event indicators can be represented visually and/or aurally in any manner that communicates certain characteristics of the respiratory event, for example, an estimated severity and/or a calculated confidence level. For example, respiratory event indicators can visually provide information based at least party on a shape, a pattern, a color, a flash of light, a number, and/or a word. As another example, respiratory event indicators can aurally provide information based at least party on a volume, a tone, a frequency, a rhythm, and/or specific words. Moreover, respiratory event indicators can present information both visually and aurally. In some embodiments, respiratory event indicators can also be presented by other methods, such as vibrations (e.g., similar to a mobile phone in vibrate mode).

Respiratory event indicators that represent both the estimated severity and the calculated confidence level can present these parameters together or separately. For example, one symbol can represent both of these parameters or one symbol can represent each of these parameters. An example rating system incorporating both the confidence level and the estimated severity is provided below in connection with FIG. 9.

FIG. 4 illustrates an example display 400 of respiratory trend data and respiratory event indicators from a respiratory monitoring system of FIG. 3. For example, the display 400 is an example of the display 320 (FIG. 3). The display 400 includes a respiratory rate trend graph 410 and a current respiratory rate indicator 420. The respiratory rate trend graph 410 can represent trends in respiratory rate data provided by the respiratory rate calculator 312 (FIG. 3) over a time window. The current respiratory rate indicator 420 can provide the most recent respiratory rate computed by the respiratory rate calculator 312 (FIG. 3).

The respiratory rate trend graph 410 can be presented along with respiratory event indicators 412, 414, 416, 418 that indicate the estimated severity and/or the calculated confidence level determined by the respiratory event detector 314 (FIG. 3). Each of the respiratory event indicators 412, 414, 416, 418 is represented by different shapes. In this embodiment, the different shapes can represent varying levels of estimated severity of the respiratory event. In addition, in this embodiment, a fill of the shape can represent the calculated confidence level of a detection of the respiratory event.

A first respiratory event indicator 412 can represent the lowest severity of the illustrated respiratory event indicators 412, 414, 416, 418. As the respiratory rate on the respiratory rate trend graph 410 decreases, the severity of the indicators can increase. This can be a result of detecting a respiratory event for a longer duration or more than one time. For example, the respiratory rate can decrease on the respiratory rate trend graph 410 due to a longer apnea event or more than one apnea event, and this in turn is reflected in the estimated severity.

As the respiratory rate decreases on the respiratory rate trend graph 410 decreases, the confidence level of detecting the respiratory event can also increase. The decrease in respiratory rate can correlate with certain respiratory events, such as apnea, and thus increase the confidence in detecting such events. The respiratory event indicators 412, 414 represented by outlines of shapes can indicate a lower confidence level than the respiratory event indicators 416, 418 that are represented by solid shapes.

With reference again to FIG. 3, the various respiratory event indicators shown can reflect different estimated severities of respiratory events. Table 1 provides an example of respiratory data from which the respiratory event detector 314 of FIG. 3 can determine an estimated severity of a detected apnea event. Based on the duration of the apnea event, the estimated severity can be represented by a point indicator. Apnea events that occur for a longer duration of time can be represented by a higher point indicator to reflect that the apnea event is more severe. For example, an apnea event of less than 10 seconds can correspond to a 4 point indicator (e.g., a 4-point star or other shape) and an apnea event of more than 30 seconds can correspond to a 12 point indicator (e.g., a 12-point star or other shape). More than one occurrence of an apnea event can also be reflected in a higher point indicator. For example, a single respiratory pause event of less than 10 seconds can correspond to a 4 point indicator and a second apnea event of less than 10 seconds can correspond to a 5 point indicator.

TABLE 1

| Parameter | Example Event | Time | | Output |
|---|---|---|---|---|
| Respiratory | Apnea | <10 | Sec | 4 Point Indicator |
| Rate (RR) | | | | |
| RR | Apnea | <10 | Sec X # | 5 Point Indicator |
| | | | Times | |
| RR | Apnea | 11-19 | Sec | 7 Point Indicator |
| RR | Apnea | 20-30 | Sec. | 9 Point Indicator |
| RR | Apnea | >30 | sec. | 12 Point Indicator |

Alternatively or in addition to an estimated severity of a respiratory event, the respiratory event detector 314 can determine a calculated confidence level in detecting the respiratory event. The confidence level of detecting the respiratory event can correlate with an occurrence of the respiratory event. The calculated confidence level can be represented a number of ways, for example, by a color and/or a pattern.

The calculated confidence level can be based on any additional indicator that a respiratory event has been properly detected. The calculated confidence level of detecting a event can be based on, for example, one or more of a duration of event, a number of occurrences of an event, a more pronounced event as determined by features of the respiratory data, a determination of noise below a predetermined threshold in the respiratory data, or other parameters calculated based on the respiratory data. Alternatively or additionally, the calculated confidence level can be based at least partly on the type of respiratory event detected. For example, in certain embodiments, certain respiratory events can be more reliably detected than others.

Table 2 provides an example of respiratory data from which a calculated confidence level can be determined. This table provides example respiratory event indicators for when the respiratory event detector 314 detects an apnea event. Based on other information related to the respiratory data provided by, for example, an acoustic sensor, the respiratory event detector 314 can represent the calculated confidence level of an apnea event by a color. As shown in Table 2, an apnea event can have a white indicator when an apnea event is detected. When the detection of the apnea event is also supported by one or more confidence indicators, the calculated confidence level can be represented by a different color. As shown in Table 2, when an apnea event is detected and a respiratory rate is below a threshold, an apnea event can be represented by a silver indicator. The silver indicator represents a higher confidence level because having a respiratory rate below a threshold makes it more likely that an apnea event has been properly detected. A second confidence indicator can further raise the confidence level of properly detecting a respiratory event. When an apnea event is detected, a respiratory rate is below a threshold, and the respiratory data has been determined to have a noise level below a threshold, an apnea event can be represented by a gold indicator. The gold indicator can represent a higher calculated confidence level than the silver or white indicators because a low noise level makes it even more likely that an apnea event was properly detected.

As also shown in Table 2, respiratory event indicators can indicate both an estimated severity of a respiratory event and a calculated confidence level in a detection of the respiratory event. The estimated severity shown in the example of Table 2 can be represented by a point indicator that can be determined based on the duration of an apnea event, as described in connection with Table 1. Some confidence indicators can be independent of the estimated severity. For example, low noise respiratory data can increase a confidence level of detecting a respiratory event regardless of the estimated severity of the respiratory event. Other confidence indicators can be related to the estimated severity of the respiratory event. For example, a lower respiratory rate threshold may be used to increase the calculated confidence level of detecting a longer, more severe apnea event. Alternatively or additionally, yet other confidence indicators can be partly independent of and partly related to the estimated severity. For example, such confidence level indicators can be independent of the estimated severity at lower levels of estimated severity and related to the estimated severity at higher levels of estimated severity.

TABLE 2

| Parameter | Example Event | Time | Output |
|---|---|---|---|
| RR | Apnea | <10 Sec | White 4 Point |
| | | | Indicator |
| RR | Apnea | <15 Sec | White 7 Point |
| | | | Indicator |
| RR | Apnea | <30 Sec | White 12 Point |
| | | | Indicator |
| RR | Apnea | <10 Sec | Silver 4 Point |
| | RR <20 | | Indicator |
| RR | Apnea | <15 Sec | Silver 7 Point |
| | RR <15 | | Indicator |
| RR | Apnea | <30 Sec | Silver 12 Point |
| | RR <10 | | Indicator |
| RR | Apnea | <10 Sec | Silver 4 Point |
| | RR <20 | | Indicator |
| | Low Noise Respiratory Data | | |
| RR | Apnea | <15 Sec | Silver 7 Point |
| | RR <15 | | Indicator |
| | Low Noise Respiratory Data | | |
| RR | Apnea | <30 Sec | Silver 12 Point |
| | RR <10 | | Indicator |
| | Low Noise Respiratory Data | | |

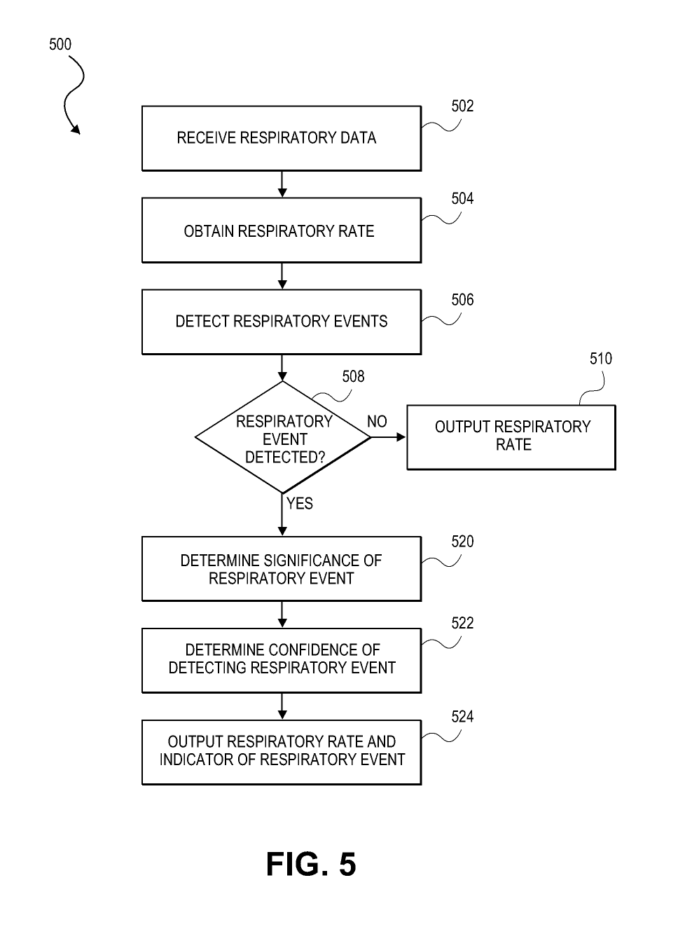

FIG. 5 illustrates an embodiment of a process 500 for displaying respiratory data. The process 500 can be used to present respiratory information to the display 400 (FIG. 4). The process 500 can be implemented by the respiratory monitoring system 300 (FIG. 3) described above or by any of the other systems described herein. Each block of the process 500 can be implemented in software, hardware, firmware, or a combination of the same. Further, one or more blocks can be implemented using separate modules, processors, or the like. Advantageously, the process 500 can receive respiratory data as an input and generate a respiratory rate and an indicator of a respiratory event as outputs. As a result, a clinician can be informed of a respiratory event and take appropriate measures to care for the patient.

At block 502 of the process 500, respiratory data is received. The respiratory data can be obtained from any respiratory sensor coupled to a patient, for example, acoustic sensors 103 (FIG. 1), 201 (FIG. 2). The respiratory data can be provided from a respiratory sensor with or without pre-processing. For example, in some embodiments, high frequency noise can be filtered out with a low pass filter.

At block 504 of the process a respiratory rate is obtained from the respiratory data. The respiratory rate can be computed, for example, by the respiratory rate calculator 312 (FIG. 3).

At block 506, respiratory events are detected, for example, as described above in reference to the respiratory event detector 314 (FIG. 3). If at decision block 508, a respiratory event is not detected, then the respiratory rate is output at block 510. The respiratory rate can then be presented on any of the displays described herein.

Alternatively, if at decision block 508 a respiratory event is detected, then the process 500 proceeds to block 520. At block 520, an estimated severity of the respiratory event is determined. This can be implemented by the any of the techniques described in connection with the respiratory event detector 314 (FIG. 3). At block 522 a confidence level in detecting the respiratory event is determined. This can be implemented by the any of the techniques described in connection with the respiratory event detector 314 (FIG. 3). As described above with reference to FIG. 3, the confidence level and the estimated severity can be related and the determination of one can be dependent on the other. Thus, in some embodiments, blocks 520 and 522 can be implemented simultaneously, in a different order, and/or provide results that are based on the other block. Yet in other embodiments, only one of an estimated significance and a confidence level are determined.

After the estimated severity and/or the confidence level are determined, at block 524 the respiratory rate and an indicator of the respiratory event are output. The indicator of the respiratory event can be any indicator described herein. The respiratory rate and the respiratory event indicator can then be presented on any of the displays described herein, for example, as shown in FIG. 4, 7, or 8.

Thus, in certain embodiments, the process 500 can provide respiratory rate information along with an indicator of a respiratory event. Advantageously, in certain embodiments, the process 500 can therefore provide a more complete representation of a patient's breathing events than providing respiratory rate data alone.

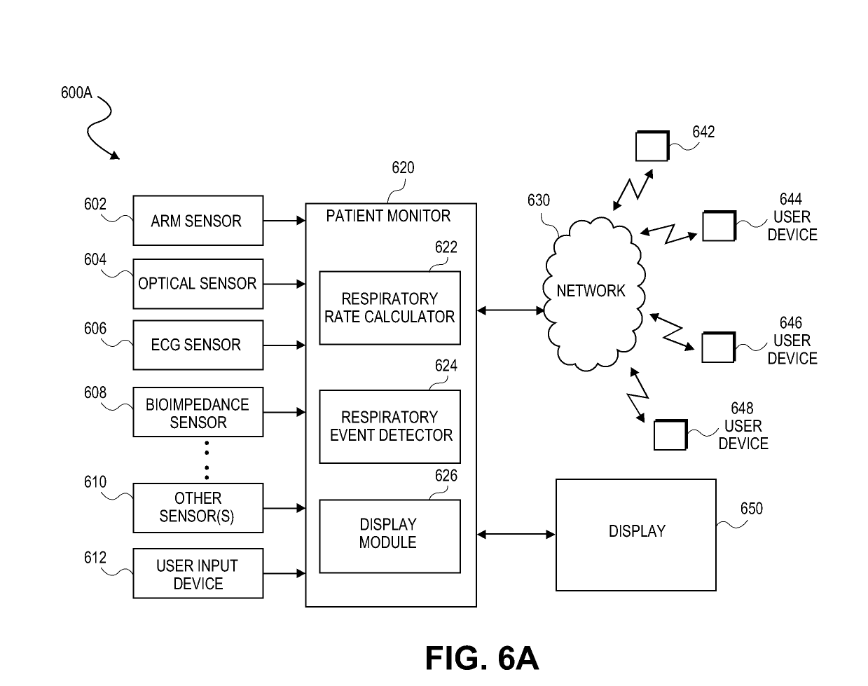

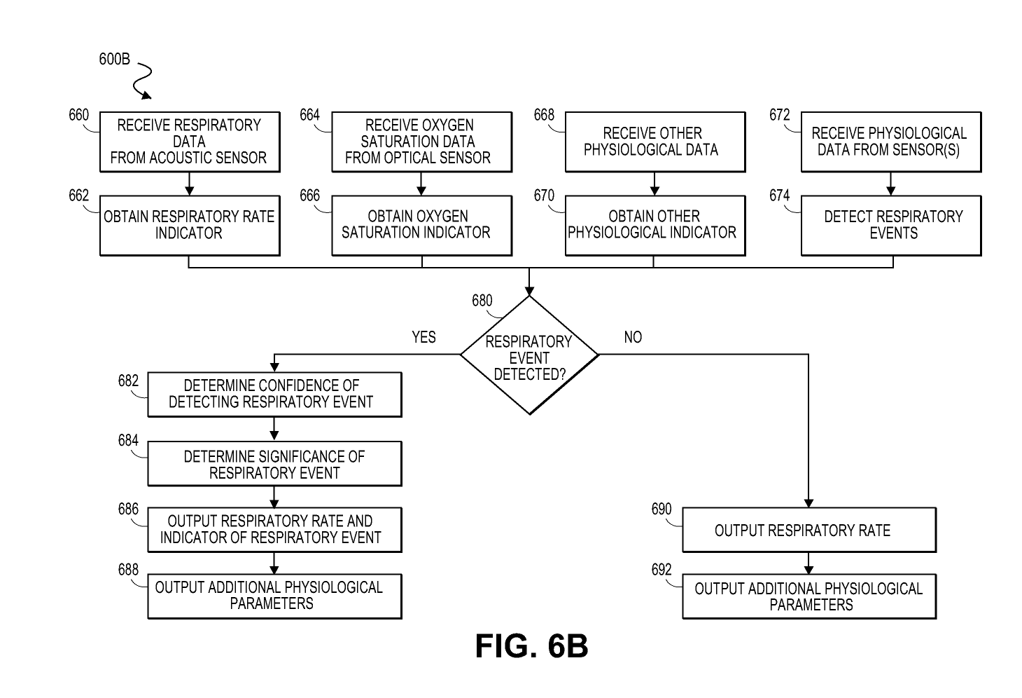

The systems, displays, and processes described above can include additional features for providing respiratory event indicators. For example, in some embodiments, a patient monitor can receive additional physiological data from additional sensors and/or user input devices. The additional physiological data can be used to obtain one or more additional parameters and/or detect respiratory events. Advantageously, in some embodiments, the one or more additional parameters can be used to determine the estimated severity and/or the calculated confidence level related to a respiratory event. FIG. 6A provides an example on one such embodiment.

FIG. 6A illustrates a respiratory monitoring system 600A. The respiratory monitoring system 600A can include a plurality of sensors 602, 604, 606, 608, 610 and a user input device 612 for obtaining physiological data. The physiological data can be provided to a patient monitor 620 for processing. The patient monitor 620 can then provide respiratory data to user devices 642, 644, 646, 648 over a network 630 and/or to a display 650.

The plurality of sensors 602, 604, 606, 608, 610 can be coupled to a patient to obtain physiological data and to provide the physiological data to the patient monitor 620. The physiological data can be used to, among other things, determine respiratory rate and detect respiratory events. An acoustic respiratory sensor 602 can include any of the features of the acoustic sensors described herein. Additional sensors 604, 606, 608, 610 can also be provided to calculate one or more additional parameters that can be used to detect a confidence level or an estimated severity of a respiratory event.

The additional sensors 604, 606, 608, 610 can provide one or more additional respiratory signals that can be used to detect any of the respiratory events described above. The one or more additional respiratory signals can be used in place of or in connection with the acoustic respiratory data described above to detect respiratory events. For example, certain respiratory events can more reliably be detected from signals provided by certain sensors. As another example, some respiratory events can be more reliably detected by a combination of signals from one or more additional sensors to the acoustic sensor.

For example, an optical sensor 604 can use spectrophotometry techniques to measure a variety of additional physiological parameters, including, for example, oxygen saturation, hemoglobin, methemoglobin, carboxyhemoglobin, other hemoglobin species, glucose, concentrations of the same, plethysmograph variability, pulse rate, perfusion, and the like. The optical sensor 604 can also provide a plethysmograph, which can be used to detect respiratory events. The optical sensor 604 can be a pulse oximetry sensor, a co-oximetry sensor, a glucose sensor, or the like.

As another example, an electrical sensor, such as an electrocardiograph (ECG) sensor 606, can be used to calculate additional parameters and provide an ECG signal for use in detecting a respiratory event. As yet another example, a bioimpedance sensor 608, such as an electrode or an ECG/defibrillator pad, can obtain bioimpedance data from a patient. The bioimpedance data can then be used to calculate additional parameters or in detecting a respiratory event. Moreover, other sensor(s) 610 can also be used to obtain additional physiological data and determine additional physiological parameters that can assist with the detection of a respiratory event and/or determine characteristics of the respiratory event.

Physiological data can also be obtained from the user input device 612. The user input device can enable a user to input patient data to the patient monitor 620. Patient data can include without limitation patient demographics and/or medical history. Patient data can be provided to the respiratory event calculator 624 for use determining a significance and/or confidence of a respiratory event. Some examples of a user device 612 include a keyboard, mouse, touch screen, or the like. The user input device 612 can be part of or separate from the patient monitor 620. In certain embodiments, one or more of the user devices 642, 644, 646, 648 can be used to provide patient data to the patient monitor 620 via the network 630.

The physiological data obtained from the plurality of sensors 602, 604, 606, 608, 610 and the user input device 612 can be provided to the patient monitor 620. The patient monitor 620 can include a respiratory rate calculator 622, a respiratory event detector 624, and a display module 626. The respiratory rate calculator 622 can implement any combination of the features of the respiratory rate calculator 312 (FIG. 3).

The respiratory event detector 624 can detect a respiratory event from physiological data obtained from one or more of the sensors 602, 604, 606, 608, 610. The respiratory event detector 624 can implement any combination of the features of the respiratory event detector 314 (FIG. 3) and detect any of the respiratory events described herein. The respiratory event detector 624 can also incorporate additional parameters obtained from one or more of the sensors 602, 604, 606, 608, 610 into a determining a calculated confidence level of detecting a respiratory event. Additionally, the respiratory event detector 624 can use additional parameters obtained from one or more of the sensors 602, 604, 606, 608, 610 and the user input device 612 in determining an estimated severity of the respiratory event. More detail about calculating the confidence level and the estimated severity will be provided later with reference to FIG. 6B and Table 3.

The display module 626 can interface with the display 650. The display module 626 can provide data to the display 650 to provide any combination of features of the example displays of FIG. 4, 8, or 9.

The patient monitor 620 can communicate with user devices 642, 644, 646, 648 via a network 630. The network 630 can include a wired and/or wireless environment. Some examples of communications protocols implemented by the network 630 can include Ethernet, WiFi (WLAN), Bluetooth, and the like. The user devices 642, 644, 646, 648 can include, but are not limited to, clinician devices, pagers, PDAs, laptops, and the like.

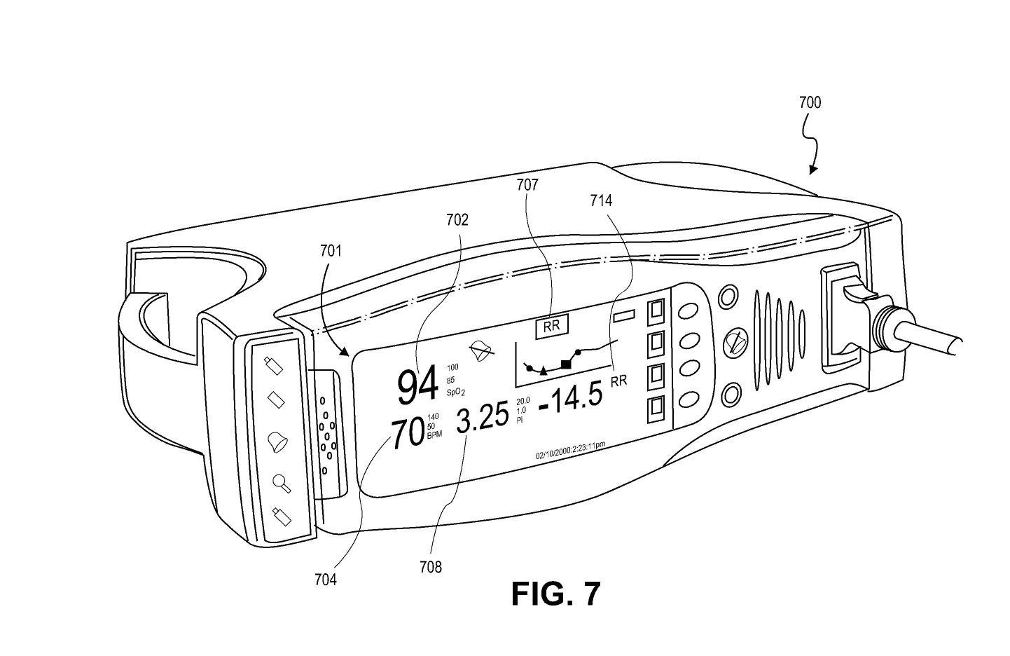

The display 650 can implement the features of any of the displays described above. The display 650 can be implemented separate from the patient monitor 620 and/or implemented as part of the patient monitor 620. For example, in one embodiment, the patient monitor 620 can be implemented as an original equipment manufacturer (OEM) board that can be incorporated with another patient monitor that includes a display. The display 650 can also display additional parameters and/or patient data. Examples of the display 650 can include the displays shown in FIGS. 7 and 8, which are described in more detail below. In addition, the display can provide indicators of a respiratory event according to the rating system described below in connection with FIG. 9.

FIG. 6B illustrates an embodiment of a process 600B for displaying respiratory data from the respiratory monitoring system of FIG. 6A. In the process 600B, physiological data can be received from multiple sources and used to obtain a respiratory rate and an indicator of a respiratory event. Advantageously, any of the physiological data obtained can be used in determining a confidence level of detecting a respiratory event or determining an estimated severity of a respiratory event.

At block 660, respiratory data can be received from an acoustic sensor, for example, the acoustic sensor 602 of FIG. 6A. Then at block 662, a respiratory rate can be obtained from the data provided by the acoustic sensor.

At block 664, oxygen saturation data can be received from an optical sensor, for example, the optical sensor 604 of FIG. 6A. Then at block 666, an indicator of oxygen saturation, such as SpO2, can be obtained. For example, SpO2 can represent oxygenation, which can be an indirect indicator of breathing. An increase in respiratory rate can correlate with an increase in SpO2. Conversely, a decrease in respiratory rate can correlate with a decrease in SpO2. Therefore, advantageously, changes in SpO2 can be useful in determining a confidence level of detecting a respiratory event. Moreover, deviations from baseline SpO2 values can also indicate an estimated severity of a respiratory event.

At block 668 other physiological data can be received from one or more of the sensors 602, 604, 606, 608, 610 or a user input device 612 of FIG. 6. The other physiological data can be used to obtain other physiological indicators at block 670. Some example parameters can include, but are not limited to, total hemoglobin (e.g., SpHb or Hbt), other forms of hemoglobin (e.g., methemoglobin or carboxyhemoglobin), plethsymograph variability (e.g., as measured by a plethysmograph variability index or PVI), a patient wellness index, patient demographic data, and patient history. Measures of hemoglobin, such as SpHb, can be reflective of bleeding, transfusion, and/or hemo-dilution/hemo-concentration. A decrease in SpHb can indicate that a respiratory system is distressed. Similarly, an increase in a PVI value can also indicate that the respiratory system is distressed. For example, when an asthma event occurs, PVI may increase. A patient wellness index can indicate a general heath of a patient, and thus respiratory event can be more severe for a patient with a lower patient wellness index. Alternatively or additionally, a patient with a low patient wellness index can be more likely to encounter a respiratory event, and therefore there can be a higher confidence in detecting a respiratory event when a patient has a decreased patient wellness index. Patient demographic data (e.g., age, weight, sex, etc.) can also factor into the confidence level in detecting a respiratory event or an estimated severity of the respiratory event. Further, a patient's known history can also be useful in detecting a respiratory event, especially history of previous occurrences of certain respiratory events.

At decision block 680, it is determined whether a respiratory event has been detected. When a respiratory event has been detected at block 680, a confidence level of detecting a respiratory event can be computed at block 682 and an estimated severity of a respiratory event can be computed at block 684. Blocks 682 and 684 can use any of the parameters described herein. One example will be discussed in detail for illustrative purposes with reference to Table 3.

TABLE 3

| Parameter | Event | Time | Output |
|---|---|---|---|
| RR | Apnea | <10 sec. | Gold 4 Point |
| SpO2 | Intermittent de-saturations | | Indicator |
| | Apnea | >15 sec. | Gold 7 Point |
| | De-sat >3 points from baseline | | Indicator |
| | Apnea | >30 sec. | Gold 12 Point |
| | De-sat >10 points from baseline | | Indicator |
| RR | Apnea | 10 Sec | SuperNova Red |
| SpO2 | DeSat>10 Points from baseline | or < Over | 4 Point |
| SpHb | Hb <1pt | X time | Indicator |
| | Apnea | >15 Sec. | SuperNova Red |
| | DeSat>10 Points from baseline | Over X | 7 Point |
| | Decrement of Hb >1pt | time | Indicator |
| | Apnea | >30 sec. | SuperNova Red |
| | DeSat>10 Points from baseline | Over X | 12 Point |
| | Hb >1pt | time | Indicator |

Table 3 provides an example of respiratory data from which a confidence level and an estimated severity of a respiratory event can be determined from one or more additional parameters. This table provides example respiratory event indicators for when the respiratory event detector 624 (FIG. 6A) detects an apnea event. As shown in Table 3, a point indicator can represent an estimated severity of a respiratory event. The point indicators shown in Table 3 increase as the duration of an apnea event increases. The additional respiratory parameters of SpO2 and SpHb can indicate a confidence level in detecting a respiratory event. For example, when an apnea event is of a certain duration, an SpO2 value indicating that oxygen de saturation is more than a certain number of points from baseline can reflect a higher confidence in detecting a respiratory event. Specific SpO2 values required for specific confidence levels can depend on the estimated severity of an event, as also shown in Table 3. Moreover, an additional parameter can indicate an even higher confidence level in detecting a respiratory event. For example, when both SpO2 and SpHb values satisfy predetermined thresholds a higher confidence level can result.

In some embodiments, a point indicator representing an estimated severity of a respiratory event can be based at least partly on patient history data. The patient history data may be obtained, for example, by the patient monitor 620 accessing an electronic file that includes patient history data. Using the patient history data, the respiratory event detector 624 can detect whether a patient has a history of experiencing a detected respiratory event, such as apnea. In some instances, the point indicator can be decreased based on the patient history data indicating that the patient has a pattern of experiencing the detected respiratory event. For example, when an apnea event is detected for a patient with a history of experiencing apnea, the point indicator can be decreased relative to a patient without a history of experiencing apnea. In other instances, the point indicator can be increased based on the patient history data indicating that the patient has a pattern of experiencing the detected respiratory event. For example, if an abnormal SpO₂ value is detected with an apnea event for patient with a history of apnea, the point indicator can be increased relative to a patient without a history of apnea.

Adjusting the point indicator based on patient history data can be advantageous because clinicians with knowledge of patient history can tend to ignore alarms related to known conditions, especially when such alarms frequently trigger. These alarms may be referred to as nuisance alarms. A patient monitor that provides a point indicator based on patient history data can better indicate a severity of a respiratory event in some instances. Consequently, such a point indicator can help a clinician better prioritize which alarms to respond to and/or provide an appropriate level of care to a detected respiratory event. The point indicator can therefore lead to improved care for patients.

One or more respiratory event indicators, such as the indicators described with reference to Table 3, can be output along with respiratory rate at block 686. Additional physiological parameters, including any of the parameters mentioned above, can also be output at block 688. FIGS. 7 and 8 provide example displays that include some of the additional physiological parameters.

If a respiratory event is not detected at decision block 680, then respiratory rate can be output at block 690. Additional physiological parameters, including any of the parameters mentioned above, can also be output at block 692.

FIG. 7 illustrates an example noninvasive multiparameter physiological monitor 700 that can implement any of the features described herein, for example, the features of the respiratory monitoring system 600A. An embodiment of the monitor 700 includes a display 701 showing data for multiple physiological parameters. For example, the display 701 can include a CRT or an LCD display including circuitry similar to that available on physiological monitors commercially available from Masimo Corporation of Irvine, Calif. sold under the name Radical™, and disclosed in U.S. Pat. Nos. 7,221,971; 7,215,986; 7,215,984 and 6,850,787, for example, the disclosures of which are hereby incorporated by reference in their entirety. However, many other display components can be used that are capable of displaying respiratory rate and other physiological parameter data along with the ability to display graphical data such as plethysmographs, respiratory waveforms, trend graphs or traces, respiratory event indicators, and the like.

The depicted embodiment of the display 701 includes a measured value of respiratory rate 714 in breaths per minute (bpm) and a respiratory rate waveform graph 706. The respiratory rate waveform graph 706 can include any of respiratory event indicators described herein. In addition, other measured blood constituents shown include SpO₂ 702, a pulse rate 704 in beats per minute (BPM), and a perfusion index 708. Many other blood constituents or other physiological parameters can be measured and displayed by the multiparameter physiological monitor 700, such as blood pressure, ECG readings, EtCO₂ values, bioimpedance values, and the like. In some embodiments, multiple respiratory rates, corresponding to the multiple input sensors and/or monitors, can be displayed.

FIG. 8 illustrates another example multiparameter physiological monitor display 801. The display 801 can output a multiparameter confidence indicator 814. The multiparameter confidence indicator 814 can be generated using any of the techniques described above.

Referring to FIG. 8, another example display 801 is shown that includes parameter data for respiratory rate, including a measured respiratory rate value 812 in breaths per minute (bpm) and a respiratory waveform graph 806 that includes respiratory event indicators. These respiratory indicators can represent an estimated severity of a respiratory event. A confidence level indicator 814 can separately indicate the confidence level in detecting the respiratory event. The display 801 also includes parameter data for SpO₂ 802, pulse rate 804 in beats per minute (BPM), and SpHb 816 in grams per deciliter (g/dL). A respiratory event multiparameter confidence indicator 814 is also depicted. In the depicted embodiment, the multiparameter confidence indicator 814 includes text that indicates that the detected respiratory event has a low multiparameter confidence level in a detection of the respiratory event.

The example displays of FIGS. 4, 7, and 8 are merely illustrative examples. Many other variations and combinations of respiratory event indicators are also possible in other implementations without departing from the spirit and/or scope of the disclosure.

The example displays of FIGS. 4, 7, and 8 can display respiratory event indicators from a number of rating systems. FIG. 9 illustrates a rating system 900. The rating system 900 is an example rating system that can simultaneously convey both a significance of a respiratory event and a confidence in the occurrence of the respiratory event using a single respiratory indicator. The rating system 900 can be implemented in any of the respiratory monitoring systems described above and displayed using any of the displays described above.

The confidence level in detecting a respiratory event can be represented by, for example, a color, a pattern, or any of the methods provided herein, for example, in connection with FIG. 3. As shown in FIG. 9, Cross Hatched indicates a lower confidence level and Super Nova indicates the highest confidence level. Each of the different confidence levels of FIG. 9 includes a different pattern inside a shape that indicates an estimated severity. An example of what each confidence level can represent is provided in Table 4.

TABLE 4

| Input/Variable Examples | Confidence Level |
|---|---|
| Any Star without Patient Demographics or History | Crosshatched |
| Single Parameter (e.g. Respiration) | Silver |
| Dual Parameter (e.g. Respiration and Oxygen | Gold |
| Saturation) | |
| Tri Parameter (e.g. Respiration, Oxygen Saturation, | Supernova (Red) |
| and Total Hemoglobin) | |

The estimated severity of a respiratory event can be represented by, for example, a shape or any of the alternatives provided herein, for example, in connection with FIG. 3. As shown in FIG. 9, the number of points on a shape can represent a corresponding estimated severity. For example, a respiratory event with an estimated severity of 7 points can be represented by a 7 point star and another respiratory event with an estimated severity of 12 can be represented by a 12 point star. Thus, the rating system 900 can be referred to as a star point system because the number points of a star can indicate an estimated severity.

Rating systems, such as the rating system 900, can be designed to be so that visually impaired people can easily decipher them. For example, someone who is color blind can decipher the rating system 900 based on the number of points on a star and the pattern inside of the star. The pattern can have either protanope, deuteranope, or tritanope color variations to assist the color blind.

### Additional Embodiments

Although some of the sensors disclosed herein, such as the acoustic sensor 103 in the sensor system 200 of FIG. 2, have been described with reference to wired implementations for illustrative purposes, it will be understood that any of the sensors described herein can communicate wirelessly to a patient monitor or other device. For instance, any of the sensors described herein can transmit and/or receive data wirelessly via any suitable communication protocol, such as WiFi, Bluetooth, or the like.

Moreover, any of the displays described herein, such as the example displays of FIGS. 4, 7, and 8, can be configured to receive user input to enable a user to select one or more respiratory event indicators. If the display is a touch screen, for instance, a clinician or other user can touch a respiratory event indicator to obtain more information. Other input methods are also possible. In response to the user selection, the one or more processors can then provide additional detail regarding the selected respiratory event indicator(s). This information can advantageously provide a clinician with a more complete picture of a patient's condition. More generally, a clinician can select any point on a respiratory trend graph to obtain additional physiological information.

For instance, in response to the user selection of a respiratory event or other point on a respiratory trend, the one or more processors can cause display of the parameter(s) and/or different indicator(s) from which a severity and/or calculated confidence were determined. Some examples of information that can be displayed can include an audio recording of a patient's respiration corresponding to the time the respiratory event occurred or within a range of time around the occurrence of the event, a display of an audio waveform corresponding to the time (or time range) when the respiratory event occurred, raw sensor data, respiratory rate values, SpO₂ values, SpHb values, the like, or any combination thereof. According to some implementations, the parameter(s) and/or the different indicator(s) can be output at block 688 of the process 600B.

In some embodiments, the patient monitor system can prompt a user, such as a clinician, for information regarding respiratory indicators shown on a display and/or enable a user to enter such information via the display. For example, the patient monitor system can prompt a clinician for feedback regarding the clinical significance of one or more particular respiratory event indicators and save the information provided by the clinician to non-transitory memory for later use. Such feedback can be useful, for example, to refine algorithms to detect a respiratory event, determine a severity of a respiratory event, calculate a confidence in detecting a respiratory event, the like, or any combination thereof. As one example, this feedback information can be used automatically by a processor to differentiate noise in data generated by a sensor from a respiratory event. As another example, feedback data regarding respiratory events can be collected and analyzed for subsequent adjustment of respiratory rate calculation algorithms or respiratory event detection algorithms.

### Terminology

The modules described herein of certain embodiments can be implemented as software modules, hardware modules, or a combination thereof. In general, the word “module,” as used herein, can refer to logic embodied in hardware or firmware or to a collection of software instructions executable on a processor. Additionally, the modules or components thereof can be implemented in analog circuitry in some embodiments.

Conditional language used herein, such as, among others, “can,” “could,” “might,” “can,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Depending on the embodiment, certain acts, events, or functions of any of the methods described herein can be performed in a different sequence, can be added, merged, or left out all together (e.g., not all described acts or events are necessary for the practice of the method). Moreover, in certain embodiments, acts or events can be performed concurrently, e.g., through multi-threaded processing, interrupt processing, or multiple processors or processor cores, rather than sequentially.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

The blocks of the methods and algorithms described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, a hard disk, a removable disk, a CD-ROM, or any other form of computer-readable storage medium known in the art. An exemplary storage medium is coupled to a processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments of the inventions described herein can be embodied within a form that does not provide all of the features and benefits set forth herein, as some features can be used or practiced separately from others. The scope of certain inventions disclosed herein is indicated by the appended claims rather than by the foregoing description. All changes which come within the meaning and range of equivalency of the claims are to be embraced within their scope.

What is claimed is:

1. A respiratory monitoring system for indicating respiratory abnormalities on a physiological monitor display, the system comprising:
an acoustic respiratory sensor configured to obtain acoustic information from a patient, the acoustic information reflecting one or more physiological parameters of the patient; and
one or more processors in communication with the acoustic respiratory sensor, the one or more processors configured to:
receive a respiratory rate value, the respiratory rate value reflecting a respiratory measurement obtained from the acoustic information averaged over a time period,
receive an indication of a respiratory abnormality occurring during the time period, and
output the respiratory rate value together with a discrete respiratory event indicator reflecting the respiratory abnormality.

2. The respiratory monitoring system of claim 1, wherein the respiratory rate value does not directly indicate an occurrence of the respiratory abnormality.

3. The respiratory monitoring system of claim 1, wherein the respiratory abnormality reflects an apnea or hypopnea event.

4. The respiratory monitoring system of claim 1, wherein the respiratory event indicator further reflects a severity of the respiratory abnormality.

5. The respiratory monitoring system of claim 4, wherein a shape of the respiratory event indicator is configured to reflect a severity of the respiratory abnormality.

6. The respiratory monitoring system of claim 4, wherein the respiratory event indicator is further configured to indicate a calculated confidence in detecting the respiratory abnormality.

7. The respiratory monitoring system of claim 1, wherein the one or more processors are further configured to receive physiological information from one or more additional physiological sensors coupled with the patient, the physiological information reflecting the respiratory abnormality.

8. The respiratory monitoring system of claim 7, wherein the one or more processors are further configured to adjust a configuration of the respiratory event indicator responsive to the physiological information.

9. The respiratory monitoring system of claim 8, wherein the physiological information comprises one or more values for one or more of the following physiological parameters: oxygen saturation, hemoglobin, and plethysmograph variability.

10. The respiratory monitoring system of claim 1, wherein the one or more processors are further configured to cause display of additional detail regarding the indication of the respiratory abnormality in response to user input.

11. A method for indicating respiratory abnormalities on a physiological monitor display, the method comprising:
by one or more processors:
receiving a respiratory rate value from a respiratory rate calculator, the respiratory rate value corresponding to respiration of a patient averaged over a time period, such that the respiratory rate value does not directly indicate abnormal respiration in the patient;
outputting the respiratory value on a physiological monitor display for presentation to a clinician;
receiving an indication of a respiratory abnormality occurring during the time period; and
outputting an indicator reflecting the respiratory abnormality on the physiological monitor display.

12. The method of claim 11, wherein the respiratory abnormality reflects a respiratory pause.

13. The method of claim 11, wherein the respiratory abnormality reflects one or more of the following conditions: apnea, hypopnea, dyspnea, bradypnea, obstruction, wheezing, striders, and ronchi.

14. The method of claim 11 further comprising indicating a confidence in an occurrence of the respiratory abnormality.

15. The method of claim 14, wherein said indicating the confidence comprises indicating a relatively higher confidence in response to receiving additional physiological information that further reflects the respiratory abnormality.

16. The method of claim 15, wherein the additional physiological information comprises oxygen saturation information.

17. The method of claim 15, wherein the additional physiological information comprises hemoglobin information.

18. The method of claim 11, further comprising generating the indicator to further reflect a length of the respiratory abnormality.

19. The method of claim 11, further comprising generating the indicator to further reflect a severity of the respiratory abnormality.

20. The method of claim 11, wherein the indicator reflects one or more of the following: a length of the respiratory abnormality, a severity of the respiratory abnormality, and a confidence in occurrence of the respiratory abnormality.

21. The method of claim 11, wherein said outputting the indicator comprises superimposing the indicator over a trend graph reflecting the respiratory rate value.
